(19) United States
(12) Patent Application Publication
(10) Pub. No.: US 2019/0208143 A1
(43) Pub. Date: Jul. 4, 2019

(54) ANGLED BORESCOPES WITH DIGITAL IMAGE ORIENTATION

(71) Applicant: Xenocor, Inc., Salt Lake City, UT (US)

(72) Inventors: Lane G. Brooks, Highland, UT (US); Christopher Joseph Pratt, American Fork, UT (US); Dennis James Muhlestein, American Fork, UT (US); John T. Langell, Salt Lake City, UT (US); Ashok C. Khandkar, Salt Lake City, UT (US)

(21) Appl. No.: 16/239,163

(22) Filed: Jan. 3, 2019

Related U.S. Application Data
(60) Provisional application No. 62/613,368, filed on Jan. 3, 2018.

(51) Int. Cl. H04N 5/262; H04N 5/225; A61B 1/05; A61B 1/00; A61B 1/313

(52) U.S. Cl. CPC H04N 5/2628; H04N 5/2253; A61B 1/05; H04N 2005/2255; A61B 1/00009; A61B 1/00045; A61B 1/3132; A61B 1/00147

(57) ABSTRACT

Borescopes, such as laparoscopes and endoscopes, configured to provide for image reorientation. In some embodiments, a portion of the borescope, such as the handle, may be rotatable with respect to another portion of the borescope, such as the shaft/tube. A sensor may be provided to translate the rotational positions of these two portions into digital data to allow an image or stream of images to be digitally rotated, preferably in real time, so that a camera module and/or image sensor may be fixed to the tube, such as positioned in a distal tip of the tube, without compromising the ability of the device to allow a surgeon to fix the rotational orientation of the images in a desired manner.

### CROSS-REFERENCE TO RELATED APPLICATIONS

This application claims the benefit under 35 U.S.C. § 119(e) of U.S. Provisional Patent Application No. 62/613,368, which was filed Jan. 3, 2018 and titled “ANGLED BORESCOPES WITH DIGITAL IMAGE ORIENTATION,” which is hereby incorporated herein by reference in its entirety.

### SUMMARY

Various embodiments of apparatus, systems, and methods are disclosed herein that relate to borescopes. In preferred embodiments, the inventive concepts disclosed herein are embodied within medical borescopes, such as laparoscopes, endoscopes, and the like.

In some preferred embodiments, the borescope may comprise a handle, a tube, and a tip at the distal end of the tube. The tip may comprise one or more light sources, such as LED lights, one or more image sensors, a lens assembly, and/or other suitable borescope components as desired. Some embodiments may further comprise a dongle, which may be communicatively coupled with the device, such as by way of wires or by being plugged into the device, such as into a port formed within the handle of the device. This dongle may comprise a memory element and a processor, which may be used to process image data from an image sensor in the device. In some embodiments, the dongle may be removably coupled with the device so that it can be coupled with a plurality of distinct laparoscopes or other borescopes. For example, the dongle may comprise a data port that may be used to couple the dongle with a plurality of distinct borescopes and/or other devices, such as a general-purpose computer. In this manner, as discussed above, data obtained from the borescope, such as usage data, may be stored in the memory element of the dongle and ultimately transferred to another computer/device following a medical procedure.

In some embodiments, the borescope may further comprise a sensor, such as a potentiometer, that may be used to detect a rotational orientation of one portion of the device with respect to another. For example, a sensor may be configured to sense a rotational position of the handle with respect to the shaft, tube, and/or tip of the borescope. The sensor may be used to process image data from the tip and reorient the image data so that the tip, shaft, and/or tube, or another suitable portion of the borescope comprising the image sensor/camera, may be rotated without resulting in rotation of the video stream or other images being output. This may allow the device to be used in a manner similar to a traditional angled laparoscope but without requiring the camera to be rotatable with respect to the tube and/or maintained in a fixed orientation at the proximal end of the device during a surgical procedure. In certain preferred embodiments, the camera/image sensor may therefore be fixedly positioned in the tube or otherwise on the shaft and the shaft, and therefore the camera/image sensor, may be rotated during operation.

The borescope may further be configured such that position/orientation data from the aforementioned sensor is used to perform digital manipulation/rotation to maintain a desired image/video stream orientation on a monitor or other display. In some embodiments, the dongle may receive the position/orientation data and may be configured to process the data and perform this manipulation/rotation to output a video stream that does not rotate with the rotation of the camera/image sensor. This may allow for the novel configurations disclosed herein that allow the camera/sensor to be fixed with respect to the tube/shaft while preserving the behavior of optical rotation to which many surgeons are accustomed.

In a more specific example of a borescope, such as a laparoscope or other medical borescope, according to some embodiments, the borescope may comprise a handle and a shaft and/or tube rotatably coupled with the handle. A tip may be positioned at a distal end of the shaft/tube and may comprise an image sensor configured to generate image data. Preferably, the image sensor is fixed with respect to the shaft/tube. The rotational sensor may be configured to detect a rotational orientation of the handle with respect to the tube/shaft or, in other embodiments, with another suitable first portion of the borescope with respect to a second portion of the borescope.

The borescope may further comprise a dongle, which may be configured to receive and process image data from the image sensor. In some embodiments, the dongle may be further configured to receive and process rotational orientation data from the rotational sensor to digitally reorient the image data.

In some embodiments, the tip may comprise a camera module, which may be at least partially or fully positioned within a lumen of the tube or may be coupled to the distal end of the shaft/tube.

Some embodiments may further comprise a rotational coupling element, such as a worm gear, configured to rotationally couple the tube/shaft with the handle or, as previously mentioned, another suitable first portion of the borescope with respect to another suitable second portion of the borescope. In some such embodiments, the rotational coupling element may be configured to limit a degree to which the tube/shaft/first portion can rotate with respect to the handle/second portion. The worm gear or other rotational coupling element may be positioned within the handle in some embodiments.

In an example of a borescope according to other embodiments, the borescope may comprise a handle and a shaft, which may comprise a lumen and may be rotatably coupled with the handle. The borescope may further comprise an image sensor configured to generate image data and a rotational sensor configured to detect a rotational orientation of the image sensor with respect to the handle and generate rotational orientation data comprising data indicative of a rotational orientation of the image sensor with respect to the handle. The image sensor may be fixedly coupled with the shaft.

The borescope, or a suitable system including the borescope, may further comprise an image processor configured to receive and process image data from the image sensor and rotational orientation data from the rotational sensor, which image processor may be configured to digitally reorient the image data using the rotational orientation data.

In some embodiments, the rotational orientation data may comprise data indicative of a rotational orientation of the handle with respect to the shaft.

Some embodiments may further comprise a camera module containing the image sensor. In some such embodiments, the camera module may be positioned at a distal end of the shaft.

Some embodiments may further comprise a dongle coupled with the borescope, which dongle may be removable from the handle or another suitable element of the borescope and/or may be configured to receive and process image data from the image sensor. In some such embodiments, the dongle may include the image processor and may therefore be configured to receive and process rotational orientation data from the rotational sensor to digitally reorient the image data.

In an example of a method for digitally reorienting image data from a borescope according to some implementations, the method may comprise generating image data using a borescope. The borescope may comprise a first portion comprising an image sensor, such as a shaft and/or tube of the borescope, and a second portion, such as a handle of the borescope, that may be rotatably coupled to the first portion. The method may further comprise rotating the first portion with respect to the second portion and sensing an orientation, such as a rotational orientation, of the first portion with respect to the second portion and using a sensed orientation of the first portion with respect to the second portion to digitally reorient the image data.

Some implementations may further comprise displaying a video stream comprising the image data, such as preferably a real-time video stream. The video stream may maintain a fixed orientation, wherein, but for the step of using a sensed orientation of the first portion with respect to the second portion to digitally reorient the image data, the video stream would rotate.

Some implementations may further comprise generating rotational orientation data comprising data indicative of a rotational orientation of the first portion of the borescope with respect to the second portion of the borescope sensor and transmitting the rotational orientation data and the image data to a dongle coupled with the borescope. The rotational orientation data and/or the image data may be processed using the dongle to digitally reorient the image data and generate digitally-reoriented image data. A video stream of the digitally-reoriented image data may then be transmitted and/or displayed, preferably in real time.

The features, structures, steps, or characteristics disclosed herein in connection with one embodiment may be combined in any suitable manner in one or more alternative embodiments.

### BRIEF DESCRIPTION OF THE DRAWINGS

The written disclosure herein describes illustrative embodiments that are non-limiting and non-exhaustive. Reference is made to certain of such illustrative embodiments that are depicted in the figures, in which:

FIG. 1 depicts a borescope system according to some embodiments;

FIG. 2 depicts the distal end of a borescope according to some embodiments;

FIG. 3 depicts the distal end of a borescope according to other embodiments;

FIG. 4 depicts the distal end of another embodiment of a borescope;

FIG. 5 depicts the distal end of a borescope according to still other embodiments;

FIG. 6 is a schematic diagram of a sensor for use in detecting a rotational position of a portion of a borescope with respect to another portion of the borescope according to some embodiments;

FIG. 7 is a cross-sectional view of a borescope according to some embodiments;

FIG. 8 is a close-up, cutaway view of the borescope of FIG. 7 illustrating the internal components of the handle; and

FIG. 9 is a schematic diagram of a borescope system according to some embodiments.

### DETAILED DESCRIPTION

It will be readily understood that the components of the present disclosure, as generally described and illustrated in the drawings herein, could be arranged and designed in a wide variety of different configurations. Thus, the following more detailed description of the embodiments of the apparatus is not intended to limit the scope of the disclosure, but is merely representative of possible embodiments of the disclosure. In some cases, well-known structures, materials, or operations are not shown or described in detail.

Various embodiments of apparatus and methods are disclosed herein that relate to borescopes and other related medical borescoping, such as laparoscopy, endoscopy, and the like. The present inventors also anticipate possible uses of the inventive teachings provided herein in connection with industrial applications, such as engine, turbine, or building inspections. In some embodiments disclosed herein, medical borescopes may be provided that mimic the behavior of more traditional angled borescopes by manipulating the images and/or video stream digitally by using a sensor in the device to maintain a desired image/video orientation during a surgical procedure.

In some preferred embodiments, the borescope may comprise a handle, a tube, and a tip at the distal end of the tube. The tip may comprise one or more light sources, such as LED lights, one or more image sensors, a lens assembly, and/or other medical borescope components. In some embodiments, the tip may further comprise a PCB and/or a memory element, such as a flash memory component or other non-volatile memory component, which may be used to store various types of data, such as the duration and/or number of uses of the device and/or model identification or calibration data, as described in U.S. patent application Ser. No. 14/958,728 titled MEDICAL BORESCOPES AND RELATED METHODS AND SYSTEMS, which was filed on Dec. 3, 2015 and is hereby incorporated herein by reference in its entirety.

As also described in the aforementioned patent application incorporated herein by reference, some embodiments may further comprise a dongle, which may be communicatively coupled with the device, such as by way of wires or by being plugged into the device, such as into a port formed within the handle of the device. This dongle may comprise a memory element and a processor, which may be used to process image data from an image sensor in the device. In some embodiments, the dongle may be removably coupled with the device so that it can be coupled with a plurality of distinct laparoscopes or other borescopes. For example, the dongle may comprise a data port that may be used to couple the dongle with a plurality of distinct borescopes and/or other devices, such as a general-purpose computer. In this manner, as discussed above, data obtained from the borescope, such as usage data, may be stored in the memory element of the dongle and ultimately transferred to another computer/device following a medical procedure.

In some embodiments, the device may further comprise a sensor that may be used to detect an orientation of a portion of the device. For example, some embodiments, may comprise a rotational position sensor configured to sense a rotational position of one portion of the device, such as the handle, with respect to another portion of the device, such as the tube and/or tip of the device. This may allow the device to be used in a manner similar to a traditional angled laparoscope but without requiring the camera to be rotatable with respect to the tube and/or maintained in a fixed orientation at the proximal end of the device during a surgical procedure.

In certain preferred embodiments, the camera/image sensor may be fixedly positioned in the tube. Thus, when the tube is rotated, the video stream/image inherently rotates with the tube. Thus, rather than using the optical rotation typically used by traditional laparoscopes, such embodiments may instead use digital rotation to mimic such optical rotation. In some such embodiments, a first portion of the device having the image sensor/camera, such as the tube, may be configured to rotate with respect to a second portion of the device, such as the handle, which may comprise a sensor, such as a rotational sensor, configured to sense a rotational orientation of at least a portion of the first portion with respect to at least a portion of the second portion. In this manner, the handle or another second portion of the device may act as the camera does in a traditional laparoscope. Thus, the doctor can maintain the handle/second portion in a fixed position while rotating the tube/first portion.

In preferred embodiments, the handle may comprise a rotational sensor configured to sense the position and/or rotational orientation of the handle with respect to the tube, which, again, may be rotatable with respect to the handle. The device may be configured such that this position/orientation data is used to perform digital manipulation/rotation to maintain a desired image/video stream orientation on a monitor or other display. In some embodiments, the dongle may receive the position/orientation data and may be configured to perform this manipulation/rotation, in some such embodiments along with the other image processing previously mentioned. Thus, in preferred embodiments, the dongle may be configured to capture a digital video stream from the camera/tip and process the raw image sensor data to convert it to a standard color HDMI or USB video stream for display on a monitor/TV or computer/tablet/phone and may also be configured with circuitry to control the LED or other light source, the exposure level of the image sensor, and/or the rotational orientation of the video stream. This digital manipulation/rotation may be used to preserve the rotational orientation between the tube and the handle, or between two other portions of the device, to allow the camera/sensor to be fixed with respect to the tube and preserve the behavior of optical rotation to which many surgeons are accustomed.

Other novel aspects of certain embodiments of borescopes are also disclosed herein, such as camera/camera module coupling methods and assemblies, methods and structures for heat dissipation, providing for increased resolution video streams, specific methods for detecting rotational position/orientation, and related improvements.

The embodiments of the disclosure may be best understood by reference to the drawings, wherein like parts may be designated by like numerals. It will be readily understood that the components of the disclosed embodiments, as generally described and illustrated in the figures herein, could be arranged and designed in a wide variety of different configurations. Thus, the following detailed description of the embodiments of the apparatus and methods of the disclosure is not intended to limit the scope of the disclosure, as claimed, but is merely representative of possible embodiments of the disclosure. In addition, the steps of a method do not necessarily need to be executed in any specific order, or even sequentially, nor need the steps be executed only once, unless otherwise specified. Additional details regarding certain preferred embodiments and implementations will now be described in greater detail with reference to the accompanying drawings.

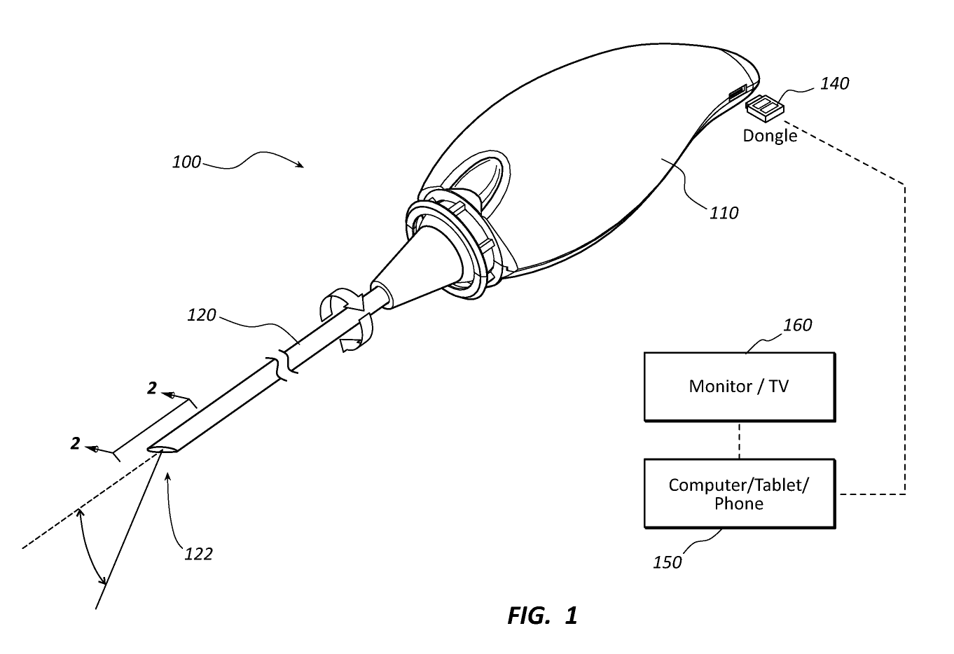

FIG. 1 depicts a borescope 100 according to some embodiments. As shown in this figure, borescope 100 comprises a handle 110, a tube 120, and a tip 122 at the distal end of tube 120. Although not visible in FIG. 1, preferably tip 122 comprises an image sensor, a lens, one or more light sources, a microprocessor, power management chips, and/or a memory component. Preferably, the tip is configured to digitize the images/video stream and control the LED or other light source illumination. Also, in the preferred embodiment of FIG. 1, tip 122 comprises an angled tip, which improves the ability to control image selection during a surgical procedure, as indicated by the angle referenced in FIG. 1. The angle of angled tip 122 may vary as desired. For example, in some preferred embodiments, this angle may be thirty degrees and therefore borescope 100 may be considered a “30-degree scope.”

As shown by the arrows in FIG. 1, preferably the tube 120 is configured to rotate with respect to the handle 110. Thus, preferably the handle comprises a sensor configured to detect a rotational orientation of the handle with respect to the tube. It is contemplated, however, that in alternative embodiments the tube may instead comprise such a rotational sensor. It is also contemplated that other portions of borescope 100 may be rotatable with respect to one another and/or comprise such a rotational sensor in still other alternative embodiments.

A dongle 140 may be communicatively coupled with handle 110. In preferred embodiments including the one depicted in FIG. 1, dongle 140 may be configured to plug into handle 110 or into another suitable portion of borescope 100.

Dongle 140 may, in turn, be communicatively coupled with a mobile general-purpose computing device 150, such as a computer/tablet/phone and/or a display 160, such as a TV or monitor. Again, although cables/wires are depicted in the figure, such as HDMI and/or USB cables, it is contemplated that any other suitable coupling techniques/structures may be used as desired. For example, in some embodiments and implementations, the dongle 140 may be unplugged from handle 110 and plugged into the mobile general-purpose computing device 150 and/or display 160 as needed.

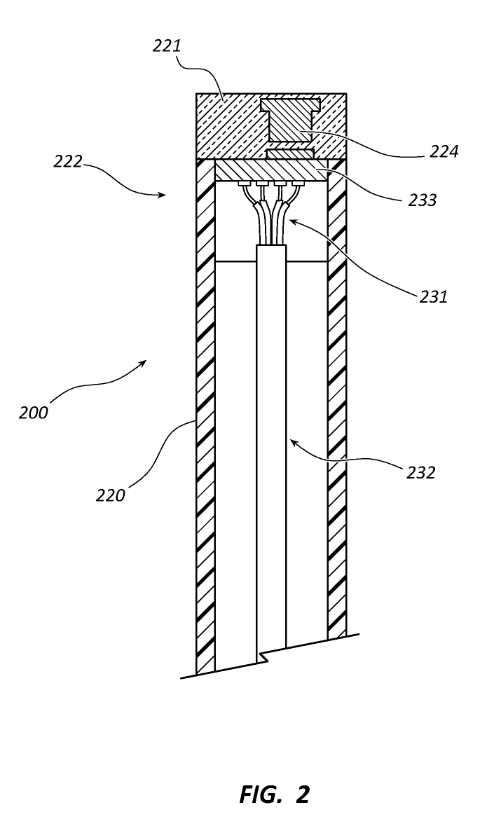

FIG. 2 illustrates a portion of a second embodiment of a borescope 200. More particularly, FIG. 2 depicts the distal end or tip 222 of tube 220 of borescope 200. In this embodiment, a camera module 221, which may comprise a lens and/or imaging assembly 224, is sealed to the distal end of tube 220. Thus, in this embodiment, the camera module 221 is external to the tube 220 and may require a seal, such as an epoxy or other adhesive, between the distal end of tube 220 and the camera module. The distal end of the tube 220 in this embodiment may comprise a PCB 233 and a potting 231 of the coupling of wires 232 with PCB 233. Although not shown in FIG. 2, wires 232 may be coupled with a dongle or a port configured to receive such a dongle.

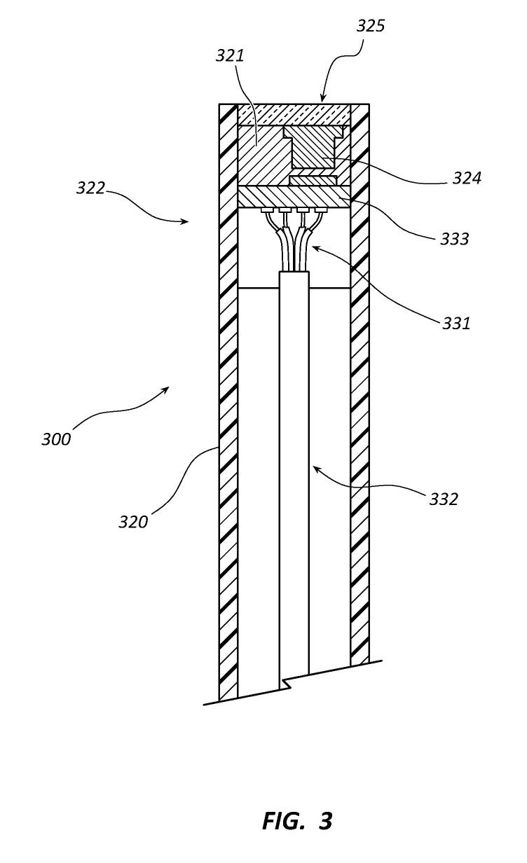

FIG. 3 depicts the distal end or tip 322 of tube 320 of an alternative embodiment of a borescope 300. In this embodiment, the camera module 321, which again may comprise a lens and/or imaging assembly 324, is positioned inside of tube 320 rather than sealed to the distal end of the tube as in borescope 200. Thus, camera module 321 may be inserted into the tube 320 and sealed in place, such as, for example, by using a suitable epoxy or other adhesive, within an adhesive reservoir 325 formed at the distal end of tube 320. This may result in an improvement of the seal relative to the design of FIG. 2. For example, even without controlled dispensing of the epoxy, an operator can fill the reservoir and visually see whether the seal fill is uniform. This may also improve the integrity and stability of the attachment of cameral module 321.

As with borescope 200, borescope 300 may further comprise a PCB 333 and a potting 331 of the coupling of wires 332 with PCB 333.

It is contemplated that, in some embodiments, the LED(s)/light source(s) and image sensor(s) may be positioned on a single PCB and encapsulated using a curable adhesive. However, this configuration may, in some embodiments, result in undesirable image sensor heating. Thus, in alternative embodiments, the LED(s)/light source(s) may be positioned on separate PCBs relative to the image sensor(s). In some embodiments, a housing, such as a lens housing, may then be used as the encapsulating feature rather than a curable adhesive.

In some preferred embodiments, a high-resolution image sensor may be used, such as, for example, an image sensor with a resolution of 1920×1080 with 1.4×1.4 μm pixels. Other embodiments may instead utilize lower resolution sensors, such as a 1280×720 image sensor with 1.75×1.75 μm pixels. In some embodiments, a plurality of image sensors and/or lens assemblies may be configured to be interchanged with one another in the borescope. However, because use of a 1080p sensor doubles the number of pixels with the same frame rate (e.g., 30 fps) relative to a 720p borescope, an unused differential pair in the cable may be provided to carry an additional serial stream so that the bandwidth requirements of the serial lines do not increase.

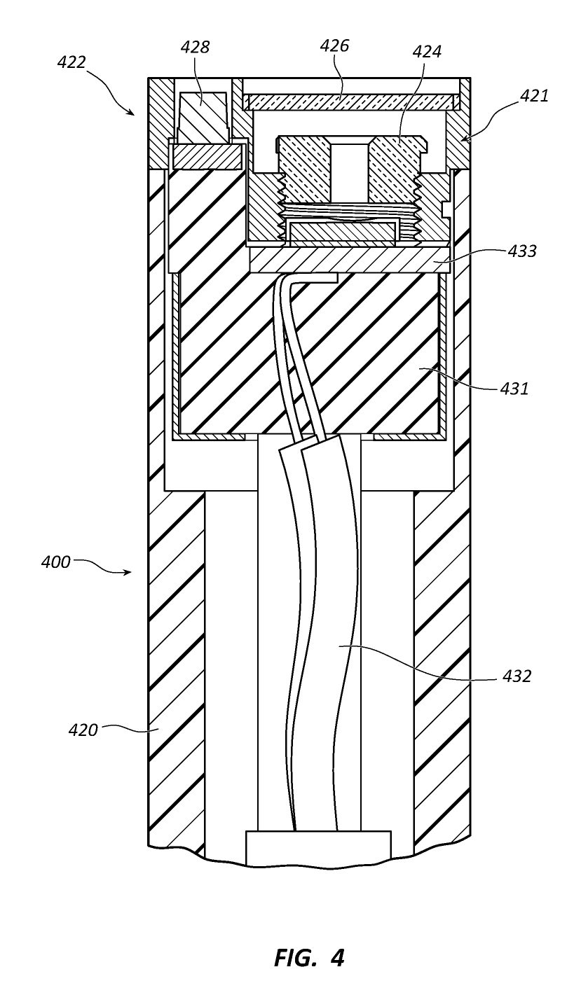

FIG. 4 depicts the distal end of another embodiment of a borescope 400 including the tip 422 of tube 420 of borescope 400. In this embodiment, a camera module 421, which, again, may comprise a lens and/or imaging assembly 424, is sealed to the distal end of tube 420. Module 421 may further comprise one or more lighting elements 428, such as LEDs or the like. However, as those of ordinary skill in the art will appreciate, such lighting elements, like other elements positioned in tip 422, may be separately coupled to borescope 400 rather than as part of a unitary assembly.

In the embodiment of FIG. 4, the camera module 421 is partially external to the tube 420 but part of camera module 421 is recessed within the distal opening of tube 420. Thus, this embodiment may also comprise a seal, such as an epoxy or other adhesive, between the distal end of tube 420 and the camera module 421. However, it is contemplated that the coupling may take place by other means and/or locations, such as from within tube 420. A cover window 426, preferably formed from a glass or other transparent material, may be positioned at the distal end of tube 420 adjacent to the imaging elements of camera module 421. Window/glass 426 may be part of camera module 421 or may be a separate element coupled to camera module 421 and/or borescope 400.

Borescope 400 further comprises a PCB 433 and a potting compound 431 or other sealant to maintain a consistent electrical coupling of wires 432 with PCB 433, which is positioned immediately adjacent and proximal of camera module 421. Wires 432 may be coupled with a dongle or a port configured to receive such a dongle, as previously mentioned.

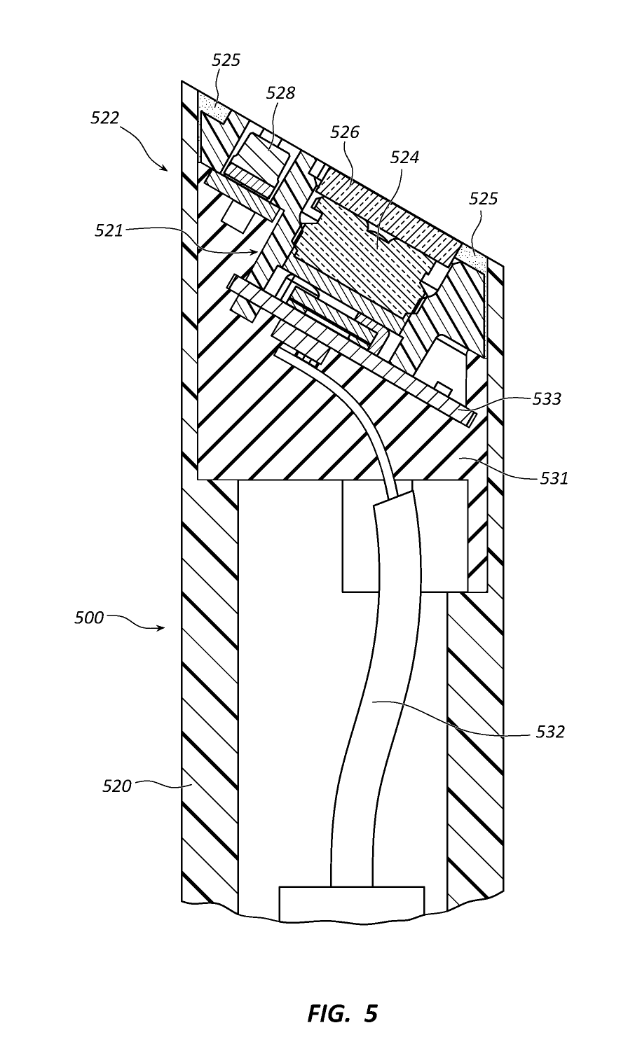

FIG. 5 depicts the distal end or tip 522 of yet another alternative embodiment of a borescope 500. Borescope 522 comprises an angled tip 522 positioned at the distal end of a lumen or tube 520. In this embodiment, the camera module 521, which again may comprise a lens and/or imaging assembly 524 and/or one or more lighting elements 528, is fully positioned inside of tube 520. Thus, in some embodiments, camera module 521 and/or any other elements as desired may be inserted into the tube 520 and sealed in place using an adhesive reservoir 525. This may result in an improvement of the seal relative to the design of FIG. 2.

As previously described in connection with various other embodiments, borescope 500 may further comprise a PCB 533 and a potting 531 or other sealant to facilitate stable coupling of wires 532 with PCB 533. As previously mentioned, the LED(s)/light source(s) 528 and image sensor(s) may be positioned on a single PCB and encapsulated using a curable adhesive or may be positioned on separate PCBs relative to the image sensor(s). In some embodiments, a housing, such as a lens housing, may then be used as the encapsulating feature rather than a curable adhesive. As with borescope 400, borescope 500 may further comprise a cover window 526, which, again, may be formed from a glass or other transparent material and may be positioned at the distal end of tube 520 adjacent to the imaging elements of camera module 521. Window/glass 526 may be part of camera module 521 or may be a separate element coupled to camera module 521 and/or borescope 500.

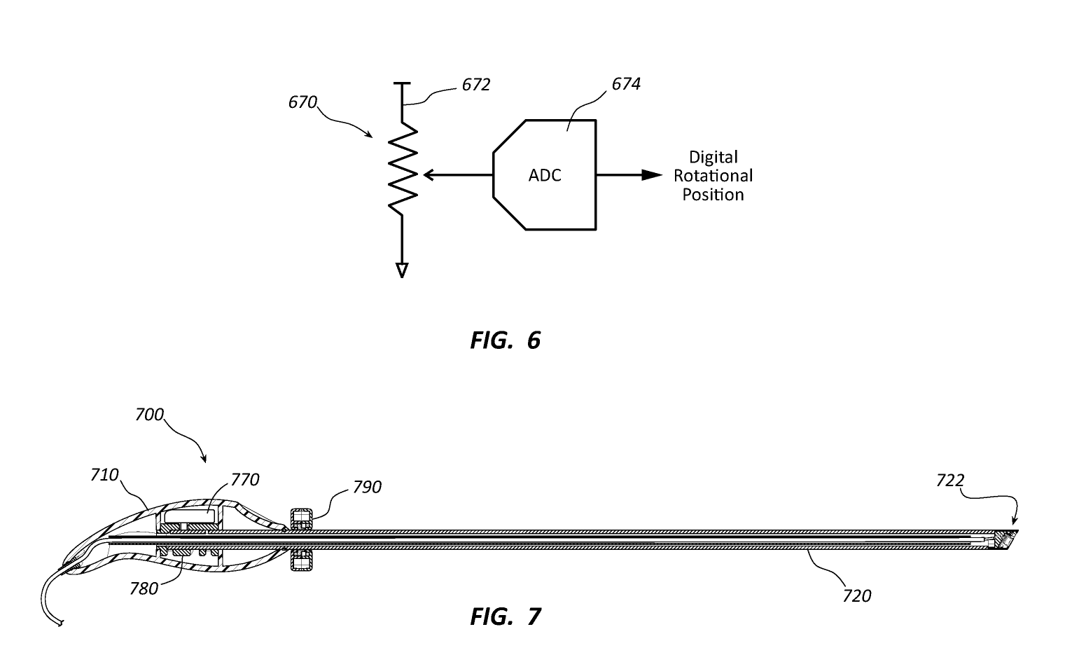

A schematic example of a rotational sensor 670 suitable for use in connection with one or more of the borescopes disclosed herein is depicted in FIG. 6. As previously mentioned, in preferred embodiments, sensor 670 may be positioned in the handle of the device, and the tube may be rotatable with respect to the handle. Preferably, the sensor 670 is configured to sense the rotational position/orientation of the tube with respect to the handle. However, as previously mentioned, alternative embodiments are contemplated in which the sensor 670 may be located elsewhere and/or other portions of the device may be rotatable with respect to one another.

As shown in FIG. 6, in some embodiments, sensor 670 may comprise a potentiometer or other voltage divider circuit 672 and an analog to digital convertor (ADC) 674. The wiper of the potentiometer 672 may be configured to move as the tube of the borescope rotates, which creates a voltage proportional to the amount/degree of rotation. This voltage may then be fed to the ADC 674, as shown in FIG. 4, to digitize the voltage and perform digital rotation of the images of the borescope, which may allow for preserving the rotational orientation of the video stream even as the tube and therefore the camera/image sensor on the distal end of the tube are rotated during a surgical procedure.

Those of ordinary skill in the art will appreciate, however, that the sensor 670 of FIG. 6 is for purposes of illustration and a variety of other sensors/solutions may also be provided for digital re-orientation of video and/or images from a borescope. For example, other possible solutions include a shaft encoder or a single-turn rotational potentiometer, which may be attached to the tube.

FIG. 7 illustrates in more detail the structure of the handle 710 of a borescope 700 and, more particularly, the coupling between the handle 710 and the tube 720, that may allow for the sensor 770 to operate in a desired manner. As shown in this figure, handle 710 may comprise a potentiometer 770 or other sensor and a rotational coupling element 780, such as a worm gear, which may be coupled with the sensor 770 to allow the tube 720 to rotate with respect to the handle 710 and to allow the rotational position to be translated into a linear position and sensed by the potentiometer 770 or other sensor. In some embodiments, tube 720 may be integrally configured with a worm gear or other rotational coupling element 780. A tip 722, which may be angled, and may comprise any of the various elements, such as lighting, imaging, memory, and/or processing elements and/or modules containing such elements, is shown at the distal end of tube 720. A rotational dial or grip 790 may also be formed adjacent to handle 710 to facilitate manual rotation of tube 720 with respect to handle 710.

In other embodiments, the shaft/tube 720 may be manufactured with an external groove, which may be used instead of a worm gear for a similar purpose. In still other embodiments, a twist potentiometer may be used instead of a slide potentiometer. Such an alternative potentiometer may be, for example, coupled directly to the shaft/tube 720, either on the proximal end or on the side via another gear mechanism. In other embodiments, a direct gear may be used to couple to a rotational potentiometer, a hall-effect sensor may be used for shaft encoding, and/or an optical shaft encoder may be used. Each of these is an example of means for sensing rotation between a first portion of a borescope and a second portion of a borescope rotatable with respect to the first portion.

In some embodiments, the sensor reading may be converted to a rotation angle by calibrating each borescope. These calibration settings may, in some embodiments, be stored in a storage element in the borescope, such as in the tip. Thus, in some embodiments, a plurality of calibration points (four, for example) may be stored and interpolation may be used for angle readings in between the calibration points.

It is contemplated that, in alternative embodiments, the ADC for the potentiometer 770 may be positioned in the tip and/or tube of the borescope. In some such embodiments, a two-conductor cable may be used to deliver the analog voltage from the potentiometer in the handle down the tube to the ADC in the tip/tube. However, the present inventors have discovered that this analog voltage may be susceptible to interference from EM radiation during electrocautery procedures. Thus, for certain applications, it may be preferable to position the ADC and the circuitry for the potentiometer 770 or other sensor in the handle 710 and instead transmit the digital signal from the handle 710 (either to the tip or directly to a dongle, for example) following conversion of the signal. This configuration may provide the benefit of elimination, or at least substantial reduction, of EM interference caused by electrocautery.

As previously mentioned, some embodiments may comprise a wire/cable that runs from the tip of the borescope through the tube and either out the handle or terminating in the handle. The present inventors have further discovered that, because in preferred embodiments the tube may be configured to rotate with respect to the handle, and because the wire/cable is preferably secured to the inside of the handle, the wire/cable must absorb the rotation over its length with appropriate strain relief. For this reason, it may be preferred to limit the ability of the handle to rotate with respect to the tube to a predetermined amount. For example, in some embodiments, the worm gear 780 or another suitable component may be used to limit such rotation to no more than a single, complete rotation. In some such embodiments, the rotation may be limited to less than a full rotation such as, for example, a quarter rotation in either direction. In alternative embodiments, the tube/shaft may be configured to rotate continuously in either the clockwise or counterclockwise directions without any limit on the degree or number of rotations.

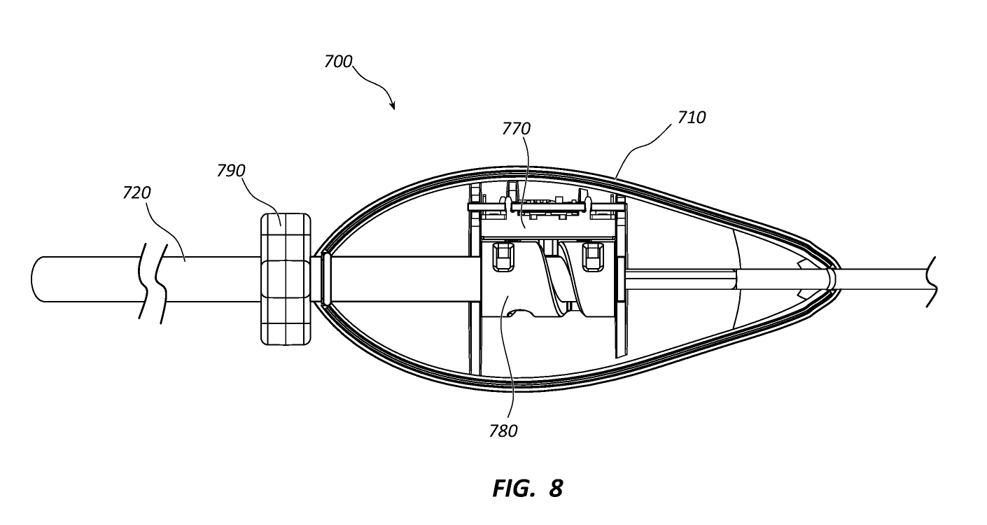

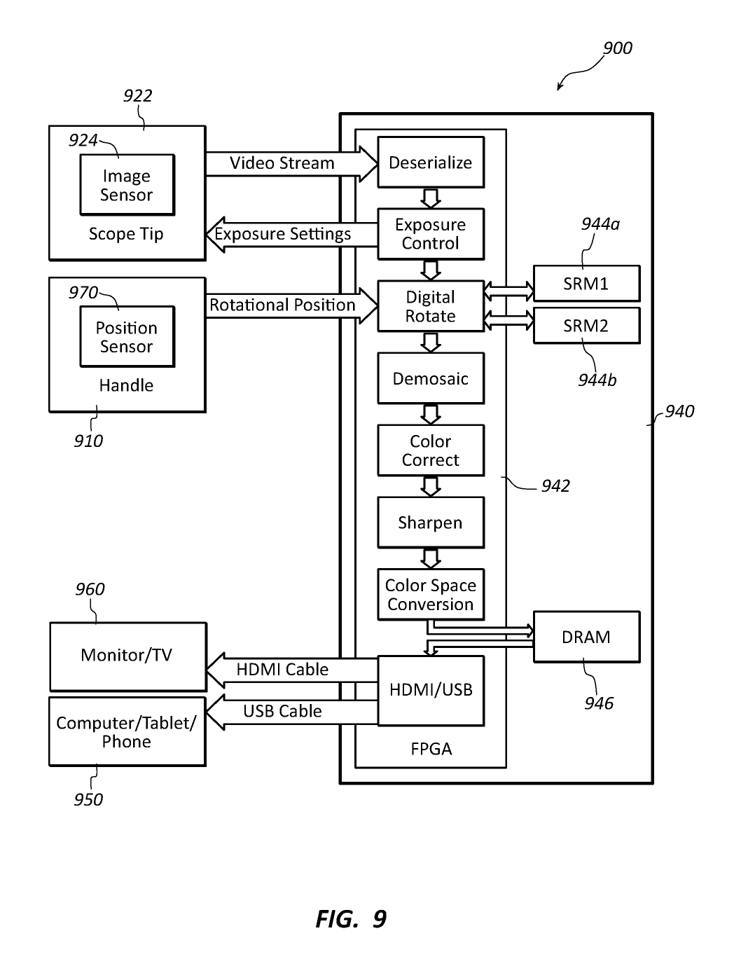

FIG. 8 is a close-up, breakaway view of the proximal end of borescope 700 including handle 710. As best illustrated in this drawing, rotational coupling element 780, which in the depicted embodiment comprises a worm gear, is operably coupled to tube/shaft 720 and handle 710 within handle 710 to allow tube/shaft 720 to rotate with respect to handle 710. As previously mentioned, in preferred embodiments, the worm gear 780 or other rotational coupling element is configured to rotationally couple the tube/shaft 720 to handle 710 so as to limit the degree to which such rotation may take place in either direction.

Sensor 770, which may comprise a potentiometer or other suitable element for sensing a degree of rotation between two elements of a borescope, is also positioned with handle 710 immediately adjacent to worm gear 780 to allow the rotational position of worm gear 780 to be translated into a linear position and sensed by the potentiometer 770 or another suitable sensor. Rotational dial or grip 790, which may comprise an annular structure extending about a desired portion of tube/shaft 720 (a portion abutting the distal portion of handle 710 in the depicted embodiment) may be fixedly coupled to tube/shaft 720 and therefore rotatably coupled to handle 720 (by virtue of the rotational coupling of tube/shaft 720 with respect to handle 720) to provide a surface to improve the ability of a surgeon/operator to rotate tube/shaft 720 with respect to handle 710. Dial/grip 790 may comprise various other features, such as bumps, knobs, grooves, a roughened surface, and/or the like to further facilitate desired

FIG. 9 is a block diagram illustrating various aspects of a preferred embodiment of a borescope 900 comprising a handle 910, a tip 922 at the end of a shaft/tube, and a dongle 940. As previously mentioned, tip 922 may comprise an image sensor 924. Although not shown in FIG. 9, various other elements may also be positioned in tip 922, such as one or more light sources, such as LED lights, one or more image sensors, a lens assembly, a PCB, and/or a memory element, such as a flash memory component or other non-volatile memory component.

As also previously mentioned, a sensor 970, such as a position sensor, may be provided. In preferred embodiments, position sensor 970 may be positioned in handle 910 and handle 910 may be rotationally coupled to the tube/shaft of the borescope 900. Thus, position sensor 970 may be configured to detect the rotational position of the handle 910 with respect to the tube/shaft and/or tip 922 so that the image(s) and/or video stream from image sensor 924 may be digitally manipulated to rotate them into a desired configuration during use.

As shown in FIG. 9, the image data, such as a video stream, may be transferred from image sensor(s) 924 in the scope tip 922 to the dongle 940, such as a Field Programmable Gate Array (FPGA) 942 of the dongle 940. The FPGA 942 may be configured to serialize the image data and apply one or more settings to the scope tip, such as exposure settings. Positional data, such as rotational position data, may be transferred from position sensor 970 to dongle 940. Digital rotation/manipulation of the image data may then be performed using the serialized image data and the rotational position data from sensor 970.

In performing digital rotation of the image data, it may be desired to achieve as low-latency rotation as possible at the full frame rate. Low latency is desired for at least two reasons. First, latency affects the ability of the surgeon to perform real-time surgery. Delay in the video stream could cause over-correction, tool misplacement, etc. Second, it may be desired to mimic the optical rotation of a traditional laparoscope, as previously mentioned. The optical rotation of traditional laparoscope does not typically introduce any latency.

In order to maintain a desired frame rate while eliminating or at least reducing latency, digital rotation may utilize a high-speed random access frame buffer. For example, under 0-degree image rotation the pixels would be read out of the frame buffer sequentially. However, in the case of a 90-degree image rotation, a pixel is read from a given row and then must access columns from non-sequential locations or from locations that are not co-located with each other. In such embodiments, access is not required to be sequential.

Although it is contemplated that some embodiments may utilize DRAM for frame buffering, doing so may introduce difficulties in providing high-speed random access for real-time image rotation. Preferred embodiments may therefore instead comprise two high-speed SRAM's in a double buffer fashion to achieve real-time digital rotation. Thus, as shown in FIG. 9, dongle 940 may comprise a first SRAM 944a and a second SRAM 944b that may, in conjunction with FPGA 642 and the positional data of sensor 970, together provide real-time or near real-time digital rotation of the image data from image sensor 924. More particularly, in some implementations, one SRAM 944a may receive the current frame while the second SRAM 944b is reading and rotating the previous frame. Then, the role of the SRAMs 944 is reversed (SRAM 944b receives the current frame and SRAM 944a reads and rotates the previous frame) when the frame is complete. This enables real-time digital rotation while adding only one frame of latency, which is acceptable and considered “real time” for most surgical applications.

In some embodiments, a dedicated Graphical Processing Unit (GPU) may be provided in place of the two, discrete SRAM units 944a and 944b. While a GPU may be able to perform real-time image rotation efficiently due to its utilization of integrated high-speed SRAM, it also adds expense. Thus, for certain applications, it may be preferable to use discrete SRAMs, as shown in FIG. 9, as a more cost-effective method of obtaining real-time, low-latency digital image rotation.

As also shown in FIG. 9, various other processing steps may be performed by dongle 940, such as demosaicing, color correction, sharpening, and/or color space conversion. One or more of these steps may be performed using a DRAM unit 946. Following digital rotation and processing of the image stream, the stream may be delivered to, for example, a display 960, such as a monitor or TV, to a mobile general-purpose computing device 950, such as a computer, tablet, or smart phone, or both. In some embodiments, the dongle may comprise common, universal, and/or non-customized display connectors such as HDMI or USB, for example, such that a common, non-customized, non-proprietary display, such as a display from a mobile general-purpose computing device may be used to display images from the device. Although cables are shown in the schematic diagram of FIG. 9, it should be understood that alternative embodiments are contemplated in which the delivery of processed image data may take place wirelessly or by way of suitable connectors, such as preferably the common, universal, and/or non-customized display connectors mentioned above, and internal wires/cables only.

It will be understood by those having skill in the art that changes may be made to the details of the above-described embodiments without departing from the underlying principles presented herein. Any suitable combination of various embodiments, or the features thereof, is contemplated.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified.

Throughout this specification, any reference to “one embodiment,” “an embodiment,” or “the embodiment” means that a particular feature, structure, or characteristic described in connection with that embodiment is included in at least one embodiment. Thus, the quoted phrases, or variations thereof, as recited throughout this specification are not necessarily all referring to the same embodiment.

Similarly, it should be appreciated that in the above description of embodiments, various features are sometimes grouped together in a single embodiment, figure, or description thereof for the purpose of streamlining the disclosure. This method of disclosure, however, is not to be interpreted as reflecting an intention that any claim require more features than those expressly recited in that claim. Rather, inventive aspects lie in a combination of fewer than all features of any single foregoing disclosed embodiment. It will be apparent to those having skill in the art that changes may be made to the details of the above-described embodiments without departing from the underlying principles set forth herein.

Likewise, benefits, other advantages, and solutions to problems have been described above with regard to various embodiments. However, benefits, advantages, solutions to problems, and any element(s) that may cause any benefit, advantage, or solution to occur or become more pronounced are not to be construed as a critical, a required, or an essential feature or element. The scope of the present invention should, therefore, be determined only by the following claims.

1. A medical borescope, comprising:
a handle;
a tube rotatably coupled with the handle;
a tip positioned at a distal end of the tube, wherein the tip comprises an image sensor configured to generate image data, and wherein the image sensor is fixed with respect to the tube;
a rotational sensor configured to detect a rotational orientation of the handle with respect to the tube; and
a dongle configured to receive and process image data from the image sensor, wherein the dongle is further configured to receive and process rotational orientation data from the rotational sensor to digitally reorient the image data.

2. The medical borescope of claim 1, wherein the tip comprises a camera module, and wherein the camera module is at least partially positioned within a lumen of the tube.

3. The medical borescope of claim 1, wherein the tip comprises a camera module, and wherein the camera module is coupled to a distal end of the tube outside of a lumen of the tube.

4. The medical borescope of claim 1, further comprising a rotational coupling element configured to rotationally couple the tube with the handle.

5. The medical borescope of claim 4, wherein the rotational coupling element is configured to limit a degree to which the tube can rotate with respect to the handle.

6. The medical borescope of claim 4, wherein the rotational coupling element comprises a worm gear.

7. The medical borescope of claim 6, wherein the worm gear is positioned within the handle.

8. The medical borescope of claim 1, wherein the rotational sensor comprises a potentiometer.

9. A borescope, comprising:
a handle;
a shaft rotatably coupled with the handle;
an image sensor configured to generate image data, wherein the image sensor is fixedly coupled with the shaft;
a rotational sensor configured to detect a rotational orientation of the image sensor with respect to the handle and generate rotational orientation data comprising data indicative of a rotational orientation of the image sensor with respect to the handle; and
an image processor configured to receive and process image data from the image sensor and rotational orientation data from the rotational sensor, and wherein the image processor is configured to digitally reorient the image data using the rotational orientation data.

10. The borescope of claim 9, wherein the shaft comprises a tube.

11. The borescope of claim 9, wherein the rotational orientation data comprises data indicative of a rotational orientation of the handle with respect to the shaft.

12. The borescope of claim 9, further comprising a camera module containing the image sensor, wherein the camera module is positioned at a distal end of the shaft.

13. The borescope of claim 9, further comprising a dongle coupled with the borescope, wherein the dongle is configured to receive and process image data from the image sensor, and wherein the dongle is further configured to receive and process rotational orientation data from the rotational sensor to digitally reorient the image data.

14. A method for digitally reorienting image data from a borescope, the method comprising the steps of:
generating image data using a borescope, wherein the borescope comprises a first portion comprising an image sensor and a second portion rotatably coupled to the first portion;
rotating the first portion with respect to the second portion;
sensing an orientation of the first portion with respect to the second portion; and
using a sensed orientation of the first portion with respect to the second portion to digitally reorient the image data.

15. The method of claim 14, further comprising displaying a video stream comprising the image data.

16. The method of claim 15, wherein the video stream maintains a fixed orientation, and wherein, but for the step of using a sensed orientation of the first portion with respect to the second portion to digitally reorient the image data, the video stream would rotate.

17. The method of claim 15, wherein the video stream comprises a real-time video stream of the image data from a procedure using the borescope.

18. The method of claim 14, wherein the first portion comprises a shaft of the borescope.

19. The method of claim 18, wherein the second portion comprises a handle of the borescope.

20. The method of claim 14, further comprising:
generating rotational orientation data comprising data indicative of a rotational orientation of the first portion of the borescope with respect to the second portion of the borescope sensor;
transmitting the rotational orientation data and the image data to a dongle coupled with the borescope;
processing the rotational orientation data and the image data using the dongle to digitally reorient the image data and generate digitally-reoriented image data; and
displaying a video stream of the digitally-reoriented image data in real-time.
